(19) United States
(12) Patent Application Publication
(10) Pub. No.: US 2004/0148648 A1
(43) Pub. Date: Jul. 29, 2004

(54) Method and system for utilizing somatic cell nuclear transfer embryos as cell donors for additional nuclear transfer

(76) Inventors: Esmail Behboodi, Worcester, MA (US); Erdogan Memili, Shrewsbury, MA (US); Yann Echelard, Jamaica Plain, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/731,542

(22) Filed: Dec. 9, 2003

Related U.S. Application Data
(60) Provisional application No. 60/432,163, filed on Dec. 10, 2002.

(51) Int. Cl. A01K 67/027

(52) U.S. Cl. 800/14; 800/21

(57) ABSTRACT

The present invention provides method to improve the efficiency of both serial nuclear transfer and the consequent production of transgenic cells and/or animals. This new method, through the use of at least a second round of nuclear transfer, will enable donor cells to correct their reprogramming more efficiently generating larger numbers of useful transgenic embryos. Because the reprogramming of the embryos generated is enhanced the current invention will allow for the production of healthier transgenic animals.

### FIELD OF THE INVENTION

The present invention relates to improved methods for the use of cells from embryos generated from somatic cell nuclear transfer methods as donors for a second round of nuclear transfer to produce transgenic animals (“re-cloning”), specifically non-human mammals. More specifically, the current invention provides improved methods of re-cloning to improve transgene expression and expression levels in transgenic animals.

### BACKGROUND OF THE INVENTION

The present invention relates generally to the field of somatic cell nuclear transfer (SCNT) and to the creation of desirable transgenic animals. More particularly, it concerns methods for generating somatic cell-derived cell lines, transforming these cell lines, and using these transformed cells through more than one round of nuclear transfer to generate transgenic non-human mammalian animal species.

Animals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, are time-consuming, costly and unreliable. Moreover, these processes are completely incapable of allowing a specific animal line from producing gene products, such as desirable protein therapeutics that are otherwise entirely absent from the genetic complement of the species in question (i.e., spider silk proteins in bovine milk).

The development of technology capable of generating transgenic animals provides a means for exceptional precision in the production of animals that are engineered to carry specific traits or are designed to express certain proteins or other molecular compounds. That is, transgenic animals are animals that carry a gene that has been deliberately introduced into somatic and/or germline cells at an early stage of development. As the animals develop and grow the protein product or specific developmental change engineered into the animal becomes apparent.

At present the techniques available for the generation of transgenic domestic animals are inefficient and time-consuming typically producing a very low percentage of viable embryos. During the development of a transgene, DNA sequences are typically inserted at random, which can cause a variety of problems. The first of these problems is insertional inactivation, which is inactivation of an essential gene due to disruption of the coding or regulatory sequences by the incoming DNA. Another problem is that the transgene may either be not incorporated at all, or incorporated but not expressed. A further problem is the possibility of inaccurate regulation due to positional effects. This refers to the variability in the level of gene expression and the accuracy of gene regulation between different founder animals produced with the same transgenic constructs. Thus, it is not uncommon to generate a large number of founder animals and often confirm that less than 5% express the transgene in a manner that warrants the maintenance of the transgenic line.

Additionally, the efficiency of generating transgenic domestic animals is low, with efficiencies of 1 in 100 offspring generated being transgenic not uncommon (Wall, 1997). As a result the cost associated with generation of transgenic animals can be as much as 250-500 thousand dollars per expressing animal (Wall, 1997).

Prior art methods have typically used embryonic cell types in cloning procedures. This includes work by Campbell et al (Nature, 1996) and Stice et al (Biol. Reprod., 1996). In both of those studies, embryonic cell lines were derived from embryos of less than 10 days of gestation. In both studies, the cells were maintained on a feeder layer to prevent overt differentiation of the donor cell to be used in the cloning procedure. The present invention uses differentiated cells. It is considered that embryonic cell types could also be used in the methods of the current invention along with cloned embryos starting with differentiated donor nuclei.

Thus, according to the present invention, multiplication of superior genotypes of mammals, including caprines, is possible. This will allow the multiplication of adult animals with proven genetic superiority or other desirable traits. Progress will be accelerated, for example, in many important mammalian species including goats, rodents, cows and rabbits. By the present invention, there are potentially billions of fetal or adult cells that can be harvested and used in the cloning procedure. This will potentially result in many identical offspring in a short period.

Thus, although transgenic animals have been produced by various methods in several different species, methods to readily and reproducibly produce transgenic animals capable of expressing the desired protein in high quantity or demonstrating the genetic change caused by the insertion of the transgene(s) at reasonable costs are still lacking. Moreover, using in vitro matured oocytes according to the current invention in a first round of cloning and generating 2-,40-, 8-, 16-, 32-cell, Morula, and Blastocyst and using these blastomeres as donor cell to do re-cloning with in vivo matured oocytes may save expenses because in vitro matured oocytes could be cheaper than in vivo generated oocytes.

Accordingly, a need exists for improved methods of nuclear transfer that will allow an increase in production efficiencies in the development of transgenic animals, particularly with regard to the development of activation of transgenic animals stably expressing desirable transgenes through the use of more than one round of nuclear transfer.

### SUMMARY OF THE INVENTION

Briefly stated, the current invention provides a method for cloning a non-human mammal through a serial nuclear transfer process comprising: obtaining desired differentiated mammalian cells to be used as a source of donor nuclei; obtaining at least one oocyte from a mammal of the same species as the cells which are the source of donor nuclei; enucleating the at least one oocyte; transferring the desired differentiated cell or cell nucleus into the enucleated oocyte; simultaneously fusing and activating the cell couplet to form a first transgenic embryo and allowing it to mature to at least a two-cell stage; then using at least one cell of that first transgenic embryo as a donor cell for at least a second round of nuclear transfer; culturing the activated second round embryo; and then either utilizing the generated cells or finally transferring the second transgenic embryo into a suitable host mammal such that the embryo develops into a fetus. Typically, the above method is completed through the use of a donor cell nuclei in which a desired gene has been inserted, removed or modified prior to insertion of said differentiated mammalian cell or cell nucleus into said enucleated oocyte. Also of note is the fact that the oocytes used are preferably matured in vitro prior to enucleation but may be matured in vivo according to the current invention.

It is also important to point out that the present invention can also be used to increase the availability of CICM cells, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimericism can be used to avoid immunological rejection among animals of the same species as well as between species.

In a preferred embodiment the methods of the current invention will improve the efficiency of nuclear transfer by improving the reprogramming of donor cells, and thereby increasing the number of embryos can be generated from a single embryo at early stage.

In an additional embodiment of the current invention matured oocytes can be manipulated in vitro to limit the use and expense of utilizing in vivo oocytes from a source animal and thereby increase the efficiency compared to use both recipients and donor cells from in vitro.

Another benefit of the methods of the current invention is through the practice of these methods cellular reprogramming will improve the heath of resulting somatic cell NT offspring as compared to offspring from a single round of NT.

An additional benefit of the current invention is that through the use of early nuclear transferred embryos as donor cell to generate a second generation of embryos the reprogramming of second generation of embryos will be enhanced.

In embodiments of the invention in which the animal is transgenic, the donor nucleus is genetically modified. The donor nucleus may contain one or more transgenes and the genetic modification may take place prior to nuclear transfer and embryo reconstitution.

### DESCRIPTION OF THE PREFERRED EMBODIMENT

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:

Blastocyst—A preimplantation embryo in placental mammals (about 3 days after fertilization in the mouse, about 5 days after fertilization in humans) of about 30-150 cells. The blastocyst stage follows the morula stage, and can be distinguished by its unique morphology. The blastocyst consists of a sphere made up of a layer of cells (the trophectoderm), a fluid-filled cavity (the blastocoel or blastocyst cavity), and a cluster of cells on the interior (the inner cell mass, or ICM). The ICM, consisting of undifferentiated cells, gives rise to what will become the fetus if the blastocyst is implanted in a uterus. These same ICM cells, if grown in culture, can give rise to embryonic stem cell lines. At the time of implantation the mouse blastocyst is made up of about 70 trophoblast cells and 30 ICM cells.

Blastula—Term to describe an early stage in the development of an embryo consisting of a hollow sphere of cells enclosing a fluid-filled cavity called the blastocoel. The term blastula sometimes is used interchangeably with blastocyst.

Caprine—Of or relating to various species of goats.

Cell Couplet—An enucleated oocyte and a somatic or fetal karyoplast prior to fusion and/or activation.

Cleavage pattern—The pattern in which cells in a very early embryo divide; each species of organism displays a characteristic cleavage pattern that can be observed under a microscope. Departure from the characteristic pattern usually indicates that an embryo is abnormal, so cleavage pattern is used as a criterion for preimplantation screening of embryos.

Clone—1) An exact genetic replica of a DNA molecule, cell, tissue, organ, or entire plant or animal. 2) An organism that has the same nuclear genome as another organism.

Embryo splitting—Separation of an early-stage embryo into two or more embryos with identical genetic makeup, essentially creating identical twins or higher multiples (triplets, quadruplets, etc.).

Embryonic stem (ES) cells—Primitive (undifferentiated ) cultured cells from the embryo that have the potential to become a wide variety of specialized cell types, (that is, are pluripotent). They are derived from the inner cell mass of the blastocyst. Embryonic stem cells are not embryos; by themselves, they cannot produce the necessary cell types, such as trophectoderm cells, in an organized fashion so as to give rise to a complete organism.

Embryonic stem (ES) cell lines—Populations of dividing cells established from embryonic stem cells and cultured in the laboratory. Within embryonic cell lines are cells that can produce more embryonic stem cells or, under conditions of differentiation, give rise to collections of cells that include most or all cell types that can be found in a postimplantation embryo, fetus, or developed organism.

Enucleation—A process whereby the nuclear material of a cell is removed, leaving only the cytoplasm. When applied to an egg, the removal of the maternal chromosomes, which are not surrounded by a nuclear membrane.

Fluorescence in situ hybridization (FISH)—A technique in which specifically designed fluorescent molecules are used to “light up” particular genes or sections of chromosomes to make them visible under a microscope. The fluorescence makes even small transgenes in the chromosomes visible.

Karyoplast—A cell nucleus, obtained from the cell by enucleation, surrounded by a narrow rim of cytoplasm and a plasma membrane.

Morula—The preimplantation embryo 3-4 days after fertilization, when it is a solid mass composed of 12-32 cells (blastomeres). After the eight-cell stage, the cells of the preimplantation embryo begin to adhere to each other more tightly, becoming “compacted”. The resulting embryo resembles a mulberry and is called a morula (Latin:morus=mulberry).

Nuclear transfer—A procedure in which a nucleus from a donor cell is transferred into an enucleated egg or zygote (an egg or zygote from which the nucleus/pronuclei have been removed). The donor nucleus can come from a Germ cell or a somatic cell.

Reprogramming—Resetting the developmental clock of a nucleus; for example, resetting the developmental state of an adult differentiated cell nucleus so that it can carry out the genetic program of an early embryonic cell nucleus, making all the proteins required for embryonic development. In somatic cell nuclear transfer, components of the recipient egg cytoplasm are thought to play an important role in reprogramming the somatic cell nucleus to carry out the functions of an embryonic nucleus.

Re-cloning or Serial Nuclear Transfer—The first step of this technique is a normal nuclear transfer, in which a nucleus is transferred into an enucleated egg, forming an embryo. In the second step, a nucleus from the resulting cloned embryo is transferred into another enucleated egg or an enucleated zygote (a fertilized egg with both male and female pronuclei removed). The second step can be repeated one or more times. This technique allows the nucleus to have two (or more) opportunities to be reprogrammed by egg cytoplasm (one during the original nuclear transfer, and more during subsequent nuclear transfers), thus potentially improving the chance of successful reprogramming of the nucleus.

Somatic cell—Any cell of a plant or animal other than a reproductive cell or reproductive cell precursor.

Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

Transgenic Organism—An organism into which genetic material from another organism has been experimentally transferred, so that the host acquires the genetic traits of the transferred genes in its chromosomal composition.

The present invention relates to a system for an increasing the number of transgenic embryos developed for nuclear transfer procedures and enhancing the reprogramming of the embryos generated. The current invention provides an improved method for the creation of transgenic animals following the use of serial nuclear transfer procedures. This capability offers an improvement in the efficiency of the creation of activated transgenic embryos for the production of live offspring in various mammalian non-human species including goats, pigs, rodents, primates, rabbits and cattle.

In addition, the present invention relates to cloning procedures in which cell nuclei derived from differentiated fetal or adult mammalian cells, which include non-serum starved differentiated fetal or adult caprine cells, are transplanted into enucleated oocytes of the same species as the donor nuclei. The nuclei are reprogrammed to direct the development of cloned embryos, which can then be transferred to recipient females to produce fetuses and offspring, or used to produce cultured inner cell mass cells (CICM). The cloned embryos can also be combined with fertilized embryos to produce chimeric embryos, fetuses and/or offspring.

Fusion of a donor karyoplast to an enucleated cytoplast, and subsequent activation of the resulting couplet are important steps required to successfully generate live offspring by somatic cell nuclear transfer. Electrical fusion of a donor karyoplast to a cytoplast is the most common method used. More importantly however, several methods of activation, and the timing of the activation steps, used in nuclear transfer methodologies to initiate the process of embryo development in numerous livestock species have been published. In mammals, while there are species differences, the initial signaling events and subsequent Ca⁺² oscillations induced by sperm at fertilization are the normal processes that result in oocyte activation and embryonic development (Fissore et al., 1992 and Alberio et al., 2001). Both chemical and electrical methods of Ca⁺² mobilization are currently utilized to activate couplets generated by somatic cell nuclear transfer. However, these methods do not generate Ca⁺² oscillations patterns similar to sperm in a typical in vivo fertilization pattern.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported.

According to the current invention, the use of recombinant somatic cell lines for nuclear transfer, and improving this procedures efficiency by increasing the number of available cells through the use of “re-cloned” embryos, not only allows the introduction of transgenes by traditional transfection methods into more transgenic animals but also increases the efficiency of transgenic animal production substantially and improves cellular reprogramming to allow normal and healthy development of transgenic embryos allowed to develop into animals.

Through the methodology and system employed in the current invention transgenic animals, goats, were generated by somatic cell nuclear transfer and were shown to be capable of producing a target therapeutic protein in the milk of a cloned animal.

According to a preferred embodiment of the current invention, embryos resulting from somatic cell NT (nuclear transfer) at various cell stages, including the 2-,40-, 8-, 16-, 32-cell, Morula, and Blastocyst cell stages can be used as cell donors to produce transgenic animals by a second round of NT (re-cloning).

In an additional embodiment of the current invention, embryos resulting from the use of in vitro matured oocytes for NT at various cell stages, including the 2-, 40-, 8-, 16-, 32-cell, Morula, and Blastocyst cell stages can be used as cell donors to produce transgenic animals through a second round of NT.

In an additional embodiment of the current invention, embryos resulting from the use of in vivo matured oocytes for NT at various cell stages, including the 2-, 40-, 8-, 16-, 32-cell, Morula, and Blastocyst cell stages can be used as cell donors to produce transgenic animals through a second round of NT.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

### MATERIALS AND METHODS

Estrus synchronization and superovulation of donor does used as oocyte donors, and micro-manipulation was performed as described in Gavin W.G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Goats.

The herds of pure- and mixed-breed scrapie-free Alpine, Saanen and Toggenburg dairy goats used for this study were maintained under Good Agricultural Practice (GAP) guidelines.

Isolation of Caprine Fetal Somatic Cell Lines.

Primary caprine fetal fibroblast cell lines to be used as karyoplast donors were derived from 35- and 40-day fetuses produced by artificially inseminating 2 non-transgenic female animals with fresh-collected semen from a transgenic male animal. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I. U. each/ml)], and were cultured in 25 cm² flasks. A confluent monolayer of primary fetal cells was harvested by trypsinization after 4 days of incubation and then maintained in culture or cryopreserved.

Sexing and Genotyping of Donor Cell Lines.

Genomic DNA was isolated from fetal tissue, and analyzed by polymerase chain reaction (PCR) for the presence of a target signal sequence, as well as, for sequences useful for sexing. The target transgenic sequence was detected by amplification of a 367-bp sequence. Sexing was performed using a zfX/zfY primer pair and Sac I restriction enzyme digest of the amplified fragments.

Preparation of Donor Cells for Multiple Rounds of Nuclear Transfer.

A transgenic female line is used for all nuclear transfer procedures. Fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 7 days. On the 7th day following the first addition of low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) 1 to 3 hours prior to fusion to the enucleated oocytes.

Oocyte Collection.

Oocyte donor does for the first round of nuclear transfer according to an embodiment of the current invention were synchronized and superovulated as previously described (Gavin W.G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml). Similarly, later rounds of nuclear transfer utilized oocytes that could have been mature in vivo or in vitro. In vivo matured oocytes are derived as explained above from donor does previously implanted with a transgenic embryo. In vitro matured oocytes are allowed to develop in vitro to a specific cell stage before they were harvested for use in a second round of nuclear transfer.

Cytoplast Preparation and Enucleation.

Oocytes with attached cumulus cells were discarded. Cumulus-free oocytes were divided into two groups: arrested Metaphase-II (one polar body) and Telophase-II protocols (no clearly visible polar body or presence of a partially extruding second polar body). The oocytes in the arrested Metaphase-II protocol were enucleated first. The oocytes allocated to the activated Telophase-II protocols were prepared by culturing for 2 to 4 hours in M199/10% FBS. After this period, all activated oocytes (presence of a partially extruded second polar body) were grouped as culture-induced, calcium-activated Telophase-II oocytes (Telophase-II-Ca) and enucleated. Oocytes that had not activated during the culture period were subsequently incubated 5 minutes in M199, 10% FBS containing 7% ethanol to induce activation and then cultured in M199 with 10% FBS for an additional 3 hours to reach Telophase-II (Telophase-II-EtOH protocol).

All oocytes were treated with cytochalasin-B (Sigma, 5 □g/ml in M199 with 10% FBS) 15 to 30 minutes prior to enucleation. Metaphase-II stage oocytes were enucleated with a 25 to 30 □m glass pipette by aspirating the first polar body and adjacent cytoplasm surrounding the polar body (˜30% of the cytoplasm) to remove the metaphase plate. Telophase-II-Ca and Telophase-II-EtOH oocytes were enucleated by removing the first polar body and the surrounding cytoplasm (10 to 30% of cytoplasm) containing the partially extruding second polar body. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in M199 for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA). Starting at between 10 to 90 minutes post-fusion, most preferably at 30 minutes post-fusion, the presence of an actual karyoplast/cytoplast fusion is determined for the development of a transgenic embryo for later implantation or use in additional rounds of nuclear transfer.

Following cycloheximide treatment, couplets were washed extensively with equilibrated SOF medium supplemented with at least 0.1% bovine serum albumin, preferably at least 0.7%, preferably 0.8%, plus 100 U/ml penicillin and 100 μg/ml streptomycin (SOF/BSA). Couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

Nuclear Transfer Embryo Culture and Transfer to Recipients.

According to a preferred embodiment of the current invention all nuclear transfer embryos were co-cultured on monolayers of primary goat oviduct epithelial cells in 50 □l droplets of M199 with 10% FBS overlaid with mineral oil. Embryo cultures were maintained in a humidified 39° C. incubator with 5% CO₂ for 48 hours before transfer of the embryos to recipient does. Recipient embryo transfer was performed as previously described.

In the experiments performed during the development of the current invention, following enucleation and nuclear transfer, the karyoplast/cytoplast couplets were incubated in equilibrated Synthetic Oviductal Fluid medium supplemented with 1% to 15% fetal bovine serum, preferably at 10% FBS, plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

The present invention allows for increased efficiency of transgenic procedures by providing for an additional generation of activated and fused transgenic embryos through the use of at least a second round of nuclear transfer procedures. The resulting embryos can be implanted in a surrogate animal or can be clonally propagated and stored or utilized. Also by combining nuclear transfer with the ability to modify and select for these cells in vitro, this procedure enhances the ability of second nuclear transfer embryos to reprogram themselves resulting in more efficient production of transgenic animals and healthier transgenic animals. In this way the current invention is more efficient than previous transgenic embryo techniques. According to the present invention, these transgenic cloned embryos can be used to produce CICM cell lines or other embryonic cell lines that have enhanced stability. Therefore, the present invention eliminates the need to derive and maintain in vitro an undifferentiated cell line that is conducive to genetic engineering techniques.

Thus, in one aspect, the present invention provides a method for cloning a mammal. In general, a mammal can be produced by a nuclear transfer process comprising the following steps:

A method for cloning a non-human mammal through a nuclear transfer process comprising:

(i) obtaining desired differentiated mammalian cells to be used as a source of donor nuclei;

(ii) obtaining at least one oocyte from a mammal of the same species as the cells which are the source of donor nuclei;

(iii) enucleating said at least one oocyte;

(iv) transferring the desired differentiated cell or cell nucleus into the enucleated oocyte;

(v) simultaneously fusing and activating the cell couplet to form a first transgenic embryo;

(vi) culturing said first transgenic embryo(es) until it reaches at least the 2-cell developmental stage; and

(vii) using at least one of the cells of said first transgenic embryo as a donor cell for the production of a transgenic animal through at least a second round of nuclear transfer so as to produce a second transgenic embryo; and

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and offspring of those mammals. The present invention is preferably used for cloning caprines. The present invention further provides for the use of nuclear transfer fetuses and nuclear transfer and chimeric offspring in the area of cell, tissue and organ transplantation.

In another aspect, the present invention provides a method for producing CICM cells. The method comprises:

(i) obtaining desired differentiated mammalian cells to be used as a source of donor nuclei;

(ii) obtaining oocytes from a mammal of the same species as the cells that are the source of donor nuclei;

(iii) enucleating said oocytes;

(iv) transferring the desired differentiated cell or cell nucleus into the enucleated oocyte;

(v) simultaneously fusing and activating the cell couplet to form a first transgenic embryo;

(vi) activating a cell-couplet that does not fuse to create a first transgenic embryo but that is activated after an initial electrical shock by providing at least one additional activation protocol including an additional electrical shock to form a second transgenic embryo;

(vii) culturing said activated first and/or second transgenic embryo until greater than the 2-cell developmental stage; and

(viii) culturing cells obtained from said second transgenic embryo to obtain CICM cells.

Also CICM cells derived from the methods described herein are advantageously used in the area of cell, tissue and organ transplantation, or in the production of fetuses or offspring, including transgenic fetuses or offspring. Differentiated mammalian cells are those cells, which are past the early embryonic stage. Differentiated cells may be derived from ectoderm, mesoderm or endoderm tissues or cell layers.

Mammalian cells, including human cells, may be obtained by well-known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures. Again the present invention is novel because differentiated cell types are used. The present invention is advantageous because the cells can be easily propagated, genetically modified and selected in vitro.

Suitable mammalian sources for oocytes include goats, sheep, cows, pigs, rabbits, guinea pigs, mice, hamsters, rats, primates, etc. Preferably, the oocytes will be obtained from caprines and ungulates, and most preferably goats. Methods for isolation of oocytes are well known in the art. Essentially, this will comprise isolating oocytes from the ovaries or reproductive tract of a mammal, e.g., a goat. A readily available source of goat oocytes is from hormonally induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields).

The stage of maturation of the oocyte at enucleation and nuclear transfer has been reported to be significant to the success of nuclear transfer methods. In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially goats, the oocyte activation period generally occurs at the time of sperm contact and penetrance into the oocyte plasma membrane.

After a fixed time maturation period, which ranges from about 10 to 40 hours, and preferably about 16-18 hours, the oocytes will be enucleated. Prior to enucleation the oocytes will preferably be removed and placed in EMCARE media containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in EMCARE media, preferably containing 7.5 micrograms per milliliter cytochalasin B, for immediate enucleation, or may be placed in a suitable medium, for example an embryo culture medium such as CRlaa, plus 10% FBS, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in EMCARE or SOF, and then viewing the oocytes under ultraviolet irradiation for less than 10 seconds. The oocytes that have been successfully enucleated can then be placed in a suitable culture medium.

In the present invention, the recipient oocytes will preferably be enucleated at a time ranging from about 10 hours to about 40 hours after the initiation of in vitro or in vivo maturation, more preferably from about 16 hours to about 24 hours after initiation of in vitro or in vivo maturation, and most preferably about 16-18 hours after initiation of in vitro or in vivo maturation.

A single mammalian cell of the same species as the enucleated oocyte will then be transferred into the perivitelline space of the enucleated oocyte used to produce the activated embryo. The mammalian cell and the enucleated oocyte will be used to produce activated embryos according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Ponimaskin et al., 2000).

Also, in some cases (e.g. with small donor nuclei) it may be preferable to inject the nucleus directly into the oocyte rather than using electroporation fusion. Such techniques are disclosed in Collas and Barnes, Mol. Reprod. Dev., 38:264-267 (1994), incorporated by reference in its entirety herein.

The activated embryo may be activated by known methods. Such methods include, e.g., culturing the activated embryo at sub-physiological temperature, in essence by applying a cold, or actually cool temperature shock to the activated embryo. This may be most conveniently done by culturing the activated embryo at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also; treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may best be effected by simultaneously, although protocols for sequential activation do exist. In terms of activation the following cellular events occur:

(i) increasing levels of divalent cations in the oocyte, and

(ii) reducing phosphorylation of cellular proteins in the oocyte.

The above events can be exogenously stimulated to occur by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation may be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

Accordingly, it is to be understood that the embodiments of the invention herein providing for an increased availability of activated and fused “re-cloned embryos” are merely illustrative of the application of the principles of the invention. It will be evident from the foregoing description that changes in the form, methods of use, and applications of the elements of the disclosed method for the improved use of transgenic embryos for Serial Nuclear Transfer are novel and may be modified and/or resorted to without departing from the spirit of the invention, or the scope of the appended claims.

### Literature Cited and Incorporated by Reference

1. Alberio R, et al., Remodeling of Donor Nuclei, DNA Synthesis, and Ploidy of Bovine Cumulus Cell Nuclear Transfer Embryos: Effect of Activation Protocol, MOL REPROD DEV 2001; 59: 371-379.

2. Baguisi A, et al., Production of Goats by Somatic Cell Nuclear Transfer, NAT BIOTECH 1999; 17: 456-461.

3. Booth P J, et al., Effect of Two Activation Treatments and Age of Blastomere Karyoplasts on In Vitro Development of Bovine Nuclear Transfer Embryos, MOL REPROD DEV 2001; 60: 377-383.

4. Bondioli K R, Westhusin M E And C R Loony, Production of Identical Bovine Offspring by Nuclear Transfer, THERIOGENOLOGY 1990; 33: 165-174.

5. Campbell, K H S, Mcwhire J, Ritchie W A And I. Wilmut. Sheep Cloned by Nuclear Transfer From a Cultured Cell Line, NATURE 1996; 380: 64-66.

6. Collas P, and Barnes FL., Nuclear Transplantation by Microinjection of Inner Cell Mass and Granulosa Cell Nuclei, MOL REPROD DEV. 1994 Jul;38(3):264-7.

7. Gavin, W. G., Gene Transfer Into Goat Embryos, in TRANSGENIC ANIMALS-GENERATION AND USE, L. M. Houdebine ed., (Harwood Academic Publishers Gmbh., 1996).

8. Kasinathan P, et al., Effect of Fibroblast Donor Cell Age and Cell Cycle on Development of Bovine Nuclear Transfer Embryos In Vitro, BIOL REPROD 2001; 64(5): 1487-1493.

9. Koo DB, et al., In Vitro Development Of Reconstructed Porcine Oocytes After Somatic Cell Nuclear Transfer. BIOL REPROD 2000; 63: 986-992.

10. Lai, L, et al, Feasibility of Producing Porcine Nuclear Transfer Embryos by Using G2/M-Stage Fetal Fibroblasts as Donors, BIOL REPROD 2001; 65: 1558-1564.

11. Park K W, et al., Developmental Potential of Porcine Nuclear Transfer Embryos Derived from Transgenic Fibroblasts Infected with the Gene for the Green Fluorescent Protein: Comparison of Different Fusion/Activation Conditions, BIOL REPROD 2001; 65: 1681-1685.

12. Ponimaskin E, et al., Sendai Virosomes Revisited: Reconstitution with Exogenous Lipids Leads to Potent Vehicles for Gene Transfer, VIROLOGY, 2000 Apr 10;269(2):391-403.

13. Stice S L, et al., Pluripotent Bovine Embryonic Cell Lines Direct Embryonic Development Following Nuclear Transfer, BIOL REPROD. 1996 Jan; 54(1):100-10.

14. Stice S L, and Keefer C L., Multiple Generational Bovine Embryo Cloning, Biol Reprod. 1993 Apr;48(4):715-9.

15. Stice S L and J M Robl, Nuclear Reprogramming in Nuclear Transplant Rabbit Embryo, BIOL REPROD 1992; 47: 636-643.

16. Wakayama T, et al., Full Term Development of Mice from Enucleated Oocytes Injected with Cumulus Cell Nuclei, NATURE 1998; 23: 369-374.

17. Westhusin M E, Pryor J H, and Bondioli K R., Nuclear Transfer In The Bovine Embryo: A Comparison Of 5-Day, 6-Day, Frozen-Thawed, And Nuclear Transfer Donor Embryos, Mol Reprod Dev. 1991 Feb;28(2):119-23.

18. Wilmut I, et al., Viable Offspring Derived From Fetal and Adult Mammalian Cells. NATURE 1997; 385: 810-813.

19. Yang X, S Jiang, A Kovacs and R H Foote, Nuclear Totipotency of Cultured Rabbit Morulae to Support Full-Term Development Following Nuclear Transfer, BIOL REPROD 1992; 47: 636-643.

20. Yong Z And L Yuqiang, Nuclear-Cytoplasmic Interaction and Development of Goat Embryos Reconstructed by Nuclear Transplantation: Production of Goats by Serially Cloning Embryos, BIOL REPROD 1998; 58: 266-269.

21. Zhang Y., and Yung Li, Biol. Reproduction 58:266-69 (1998).

22. Zou X, et al., Production of Cloned Goats from Enucleated Oocytes Injected with Cumulus Cell Nuclei or Fused with Cumulus Cells, CLONING 2001; 3 (1): 31-37.

What is claimed is:

1. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) culturing said first transgenic embryo(es) until it reaches at least the 2-cell developmental stage; and
(vii) using at least one of the cells of said first transgenic embryo as a donor cell for the production of a transgenic animal through at least a second round of nuclear transfer so as to produce a second transgenic embryo; and

2. The method of claim 1, wherein said donor differentiated mammalian cell to be used as a source of donor nuclei or donor cell nucleus is from mesoderm.

3. The method of claim 1, wherein said donor differentiated mammalian cell to be used as a source of donor nuclei or donor cell nucleus is from endoderm.

4. The method of claim 1, wherein said donor differentiated mammalian cell to be used as a source of donor nuclei or donor cell nucleus is from ectoderm.

5. The method of claim 1, wherein said donor differentiated mammalian cell to be used as a source of donor nuclei or donor cell nucleus is from fetal somatic tissue.

6. The method of claim 1, wherein said donor differentiated mammalian cell to be used as a source of donor nuclei or donor cell nucleus is from fetal somatic cells.

7. The method of claim 1, wherein said donor differentiated mammalian cell to be used as a source of donor nuclei or donor cell nucleus is from a fibroblast.

8. The method of claim 1, wherein said donor differentiated mammalian cell to be used as a source of donor nuclei or donor cell nucleus is from an ungulate.

9. The method of either claim 1 or 8, wherein said donor cell or donor cell nucleus is from an ungulate selected from the group consisting of bovine, ovine, porcine, equine, caprine and buffalo.

10. The method of claim 1, wherein said donor differentiated mammalian cell to be used as a source of donor nuclei or donor cell nucleus is from an adult non-human mammalian somatic cell.

11. The method of claim 1, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

12. The method of claim 1, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

13. The method of claim 1, wherein said at least one oocyte is matured in vivo prior to enucleation.

14. The method of claim 1, wherein said at least one oocyte is matured in vitro prior to enucleation.

15. The method of claim 1, wherein said non-human mammal is a rodent.

16. The method of claim 1, wherein said donor differentiated mammalian cell to be used as a source of donor nuclei or donor cell nucleus is a non-quiescent somatic cell or a nucleus isolated from said non-quiescent somatic cell.

17. The method of either claim 1 or 8, wherein the fetus develops into an offspring.

18. The method of claim 1, wherein said at least one oocyte is enucleated about 10 to 60 hours after initiation of in vitro maturation.

19. The method of claim 1, wherein a desired gene is inserted, removed or modified in said differentiated mammalian cell or cell nucleus prior to insertion of said differentiated mammalian cell or cell nucleus into said enucleated oocyte.

20. The resultant offspring of the methods of claim 1 or 19.

21. The resultant offspring of claim 19 further comprising wherein the offspring created as a result of said nuclear transfer procedure is chimeric.

22. The method of claim 1, wherein cytocholasin-B is used in the cloning protocol.

23. The method of claim 1, wherein cytocholasin-B is not used in the cloning protocol.

24. The method of claim 1, wherein said differentiated mammalian cells to be used as a source of donor nuclei is from an in vitro matured oocyte.

25. The method of claim 24 wherein the cells of said first transgenic embryo are utilized for the generation of at least one said second transgenic embryo when said first transgenic embryo is at most at the 40-cell stage of development.

26. The method of claim 24 wherein the cells of said first transgenic embryo are utilized for the generation of at least one said second transgenic embryo when said first transgenic embryo is at the 32-cell stage of development.

27. The method of claim 24 wherein the cells of said first transgenic embryo are utilized for the generation of at least one said second transgenic embryo when said first transgenic embryo is at the 16-cell stage of development.

28. The method of claim 24 wherein the cells of said first transgenic embryo are utilized for the generation of at least one said second transgenic embryo when said first transgenic embryo is at the 8-cell stage of development.

29. The method of claim 1, wherein said differentiated mammalian cells to be used as a source of donor nuclei is from an in vivo matured oocyte.

30. The method of claim 29 wherein the cells of said first transgenic embryo are utilized for the generation of at least one said second transgenic embryo when said first transgenic embryo is at most at the 40-cell stage of development.

31. The method of claim 29 wherein the cells of said first transgenic embryo are utilized for the generation of at least one said second transgenic embryo when said first transgenic embryo is at the 32-cell stage of development.

32. The method of claim 29 wherein the cells of said first transgenic embryo are utilized for the generation of at least one said second transgenic embryo when said first transgenic embryo is at the 16-cell stage of development.

33. The method of claim 29 wherein the cells of said first transgenic embryo are utilized for the generation of at least one said second transgenic embryo when said first transgenic embryo is at the 8-cell stage of development.

34. The method of claim 1, further comprising transferring said second transgenic embryo into a host mammal such that the embryo develops into a fetus.

35. The resultant offspring of the methods of claim 24 or 29.
